(19) United States
(12) Patent Application Publication
(10) Pub. No.: US 2023/0358422 A1
(43) Pub. Date: Nov. 9, 2023

(54) AIR PURIFIER

(71) Applicant: ACCO Brands Corporation, Lake Zurich, IL (US)

(72) Inventors: Dominic Menoni, Lake Zurich, IL (US); Ryan Himmelblau, Wheeling, IL (US); Eric Frydendall, Westminster, CO (US)

(21) Appl. No.: 17/737,303

(22) Filed: May 5, 2022

(51) Int. Cl. A61L 9/20; A61L 2/10

(52) U.S. Cl. CPC A61L 9/20; A61L 2/10; A61L 2209/14; A61L 2209/12; A61L 2202/11

(57) ABSTRACT

An air purifier may include a filter chamber having a recessed panel. The recessed panel may include openings, a plurality of UV lighting elements, and a plurality of reflectors disposed on, above, below, and/or within the recessed panel to provide a more even distribution of radiation across a surface of a filter. In some embodiments the plurality of reflectors may take the shape of a hyperbolic cone. The plurality of reflectors may be disposed above and/or adjacent to the plurality of UV lighting elements. The plurality of UV lighting elements may be disposed below or within the recessed panel. The plurality of UV lighting elements may include lighting elements which emit Ultraviolet (UV) and/or Ultraviolet C (UVC) wavelength light, such as certain light emitting diodes (LED) light sources to irradiate a surface of the filter.

### TECHNICAL FIELD

This disclosure relates generally to air cleaners or purifiers, and more specifically, to air purifiers with one or more filters for removing contaminates from the air.

### BACKGROUND

Air purifiers help remove contaminants such as particulates and/or impurities from ambient air. Air purifiers often use air filters to remove some of the contaminants from the air. Air filters may collect and store these contaminants on the surface thereof and/or throughout the filter. Over time, use of such air filters typically causes these contaminants to travel towards an inner surface of the air filter. Accordingly, manufacturers of air purifiers often include irradiation elements adjacent to the air filter to irradiate the air filter to inactivate microorganisms.

These irradiation elements commonly are placed adjacent to the air filter to allow for the radiation to reach the air filter. Current designs including irradiation elements often unevenly irradiate the surface of the air filter such that some areas on the air filter surface receive higher doses of light, and in turn more irradiation, while other areas receive lower doses of light, and in turn less irradiation.

### BRIEF DESCRIPTION OF THE DRAWINGS

Disclosed herein are embodiments of systems and apparatuses pertaining to providing an air purifier including reflective surfaces to irradiate an air filter. This description includes drawings, wherein:

FIG. 1A is an exploded view of an air purifier with multiple reflective and lighting elements in accordance with various embodiments.

FIG. 1B is an exploded view of an air purifier with multiple reflective and lighting elements in accordance with various embodiments.

FIG. 2A is a bottom cross-sectional perspective view of a reflector disposed above a lighting element in accordance with various embodiments.

FIG. 2B is a bottom cross-sectional perspective view of the reflector disposed above the lighting element of FIG. 2A.

FIG. 3A is a top perspective view of a reflector in accordance with various embodiments.

FIG. 3B is a top perspective view of a UV light component in accordance with various embodiments.

FIG. 4 is a graph illustrating reflectance of certain materials across different wavelengths of light in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments. Also, common but well-understood elements that are useful or necessary in a commercially feasible embodiment are often not depicted to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Described herein are systems, apparatus, and methods that are useful for removing contaminants from air. These teachings also may assist with improving the air purifier and the components thereof by, e.g., improving filter performance, improving air purifier unit efficiency, and/or extending the life of the filter, among other advantages. In some embodiments, a system includes an air purifier housing and a removable panel with a filter assembly disposed in a filter chamber where the filter chamber has UV lighting components configured to provide a relatively continuous and/or evenly disposed or diffused irradiation onto one or more components of the filter assembly.

Whereas an excessive dose of UV light exposure can damage a filter, these teachings help avoid filter damage. Further, often a difference between a shape of a filter and a lighting element or light source results in uneven distribution of radiation. For example, a portion of a filter closest to a light source may receive a very high dose of UV irradiation that might damage the filter over time, whereas filter portions farther away from the UV light may not receive sufficient irradiation to treat contaminates captured in the filter. This issue may be particularly acute in portions of a filter having structure with varying depths, such as, e.g., deep pleats or other variated surface structure. While current solutions to the uneven irradiation in some air purifiers are often bulky, somewhat ineffective, environmentally wasteful, and/or expensive, these teachings incorporate lighting elements and a plurality of reflectors and/or reflective surfaces designed to provide relatively evenly dispersed irradiation to filter elements.

Generally speaking, pursuant to these various embodiments and systems described herein may be used to provide an air purifier with lighting elements and reflectors to provide relatively even irradiation of a filter disposed within a filter chamber. In one illustrative approach, the air purifier includes a filter chamber having a recessed panel, the recessed panel including openings, a plurality of UV lighting elements, and a plurality of reflectors disposed on, above, below, and/or within the recessed panel to provide a more even distribution of radiation across a surface of the filter. In some embodiments the plurality of reflectors may take the shape of a hyperbolic cone. The plurality of reflectors may be disposed above and/or adjacent to the plurality of UV lighting elements. The plurality of UV lighting elements may be disposed below or within the recessed panel. The plurality of UV lighting elements may include lighting elements which emit Ultraviolet (UV) and/or Ultraviolet C (UVC) wavelength light, such as certain light emitting diode (LED) light sources to irradiate a surface of the filter. In configurations utilizing LED light sources, the light sources have a typical design life similar to that of the air purifier, such that users are not required to handle cumbersome and/or expensive replacement tasks. In addition, in some configurations, the filter chamber further includes one or more reflective panels or surfaces, such as, e.g., along a perimeter or wall thereof.

In some configurations the air purifier may include a housing having the recessed panel disposed inward from an outer surface of the housing. Securement geometry may be included for receiving and securing a removable panel to the outer surface of the housing. An air blowing unit may be disposed within the housing. The filter may be disposed between the air blowing unit and the removable panel. The filter chamber may be disposed and/or defined between the removable panel and the recessed panel. The filter chamber may include at least one peripheral reflective surface and an array of UV light components. Each UV light component may include the UV lighting element and one or more corresponding reflectors. The recessed panel may include openings to permit light emitted from the UV lighting element to enter the filter chamber which may be reflected and/or diffused off at least one of the peripheral reflective surfaces and/or the reflectors to be directed towards and irradiate the filter.

In some configurations the present disclosure may be applicable to other air treatment systems, such as for example, humidifiers. In these configurations, the UV light components and at least one peripheral reflective surface may be utilized to reflect and diffuse light across a water source, such as a water conduit or tank holding water, to irradiate the water source.

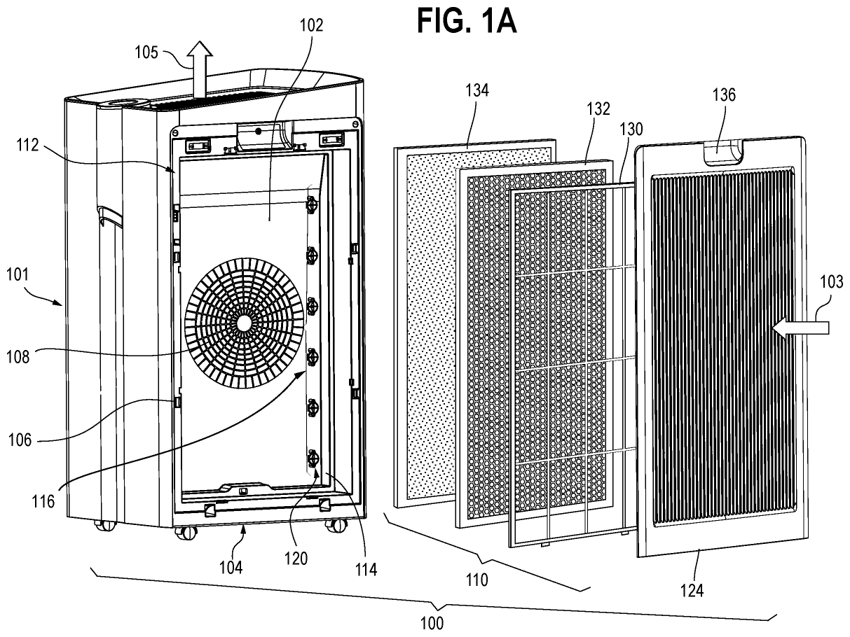

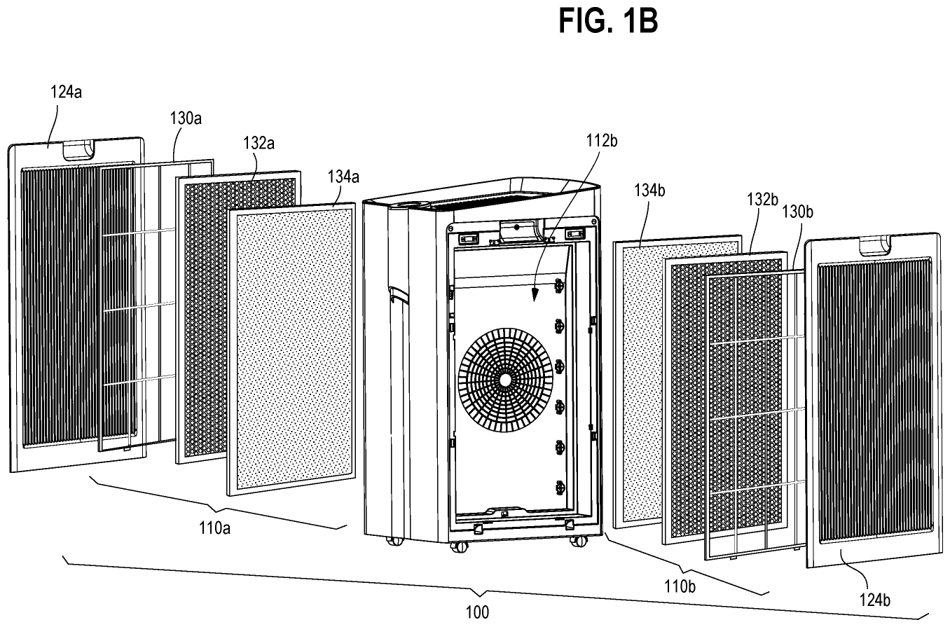

Referring now to the drawings, wherein identical numerals indicate the same elements throughout the figures. Referring to FIGS. 1A and 1B, illustrative embodiments of an air purifier 100 are provided in accordance with some embodiments. The air purifier 100 operates by drawing air in, as illustrated by arrow 103, through a removable panel 124 on a side of the air purifier 100. The removable panel 124 may include openings or slots to allow air to pass through the removable panel 124. The air may travel through the removable panel 124, through a filter assembly 110 and into a blowing unit 108 disposed within the housing 101. The blowing unit 108 may expel substantially purified air out of the air purifier 100 (as illustrated by arrow 105). While a first side of air purifier 100 is illustrated with a filter chamber 112 and filter assembly 110 disposed therein, at least a second side of the air purifier 100 also may have another, filter chamber 112 and filter assembly 110 disposed thereon, as illustrated in FIG. 1B. Thus, the air purifier 100 may filter air coming into the unit from opposing and/or adjacent sides thereof.

The removable panel 124 may be received and/or secured to a housing 101 of the air purifier 100 by securement geometry 106. As used herein, the securement geometry may include, e.g., a variety and/or a plurality of magnetic fasteners and/or mechanical fasteners, latches, snap fit, friction fit, and/or press fit configurations, among others. In addition, the removable panel 124 may include a handle or grasping surface 136 that permits a user to manually grip a portion of the removable panel 124 and pull the removable panel 124 away from a remainder of the unit housing 101 to thereby expose the filter assembly 110. The air purifier 100 may include an outer surface 104 of the housing 101. The outer surface 104 may be defined by the surface of the removable panel 124 when the removable panel 124 is secured to the housing 101. The housing 101 may also include at least one recessed panel 102 disposed inward from the outer surface 104.

The air purifier 100 may include a filter chamber 112, formed via the recessed panel 102. In some configurations, the filter chamber 112 is defined, in part, by the recessed panel 102 and the removable panel 124. During operation, the filter assembly 110 may be disposed within the filter chamber 112. The air purifier 100 may include more than one outer surface 104, removable panel 124, filter assembly 110, and filter chamber 112, as illustrated in FIG. 1B.

In one illustrative approach, the filter chamber 112 includes at least one peripheral reflective surface 114 and an array of UV light components 116. As shown in FIG. 1A, the reflective surface 114 is disposed along a sidewall of the filter chamber 112 between the recessed panel 102 and the outer surface 104.

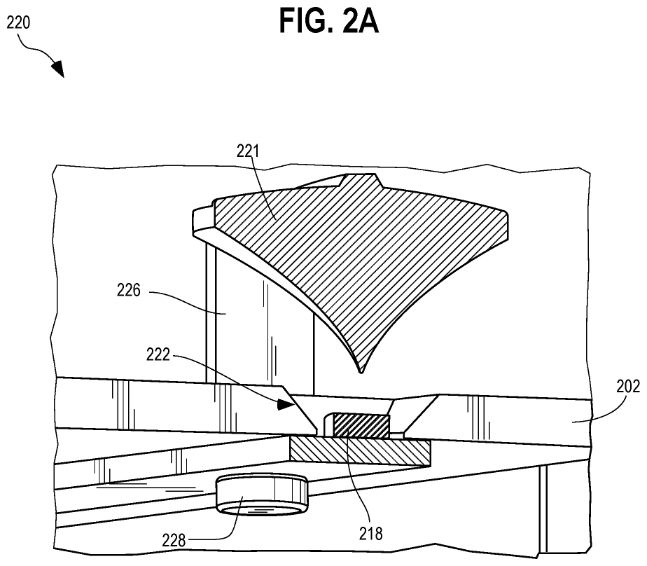

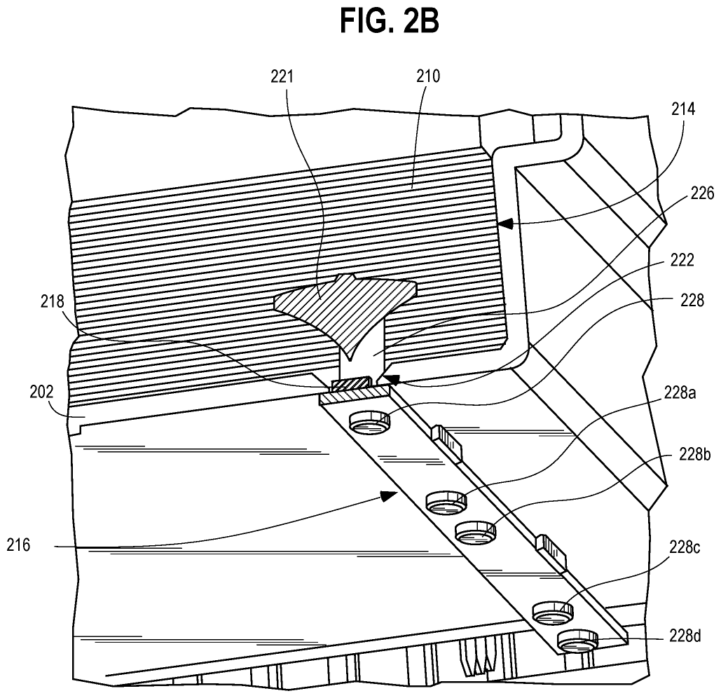

In some configurations, an exemplary UV light component 120 includes one or more UV lighting elements and one or more discrete reflectors, discussed in more detail below with reference to FIGS. 2A and 2B, in the array of UV light components 116. In some configurations, the UV lighting element emits or produces UV light, such as, e.g., UVC wavelength light. In addition, one or more discrete reflectors may correspond to the UV lighting elements. The UV light components 120 may be oriented to direct light towards the filter assembly 110. In one illustrative embodiment, there are twenty UV light components. In one illustrative embodiment, there are twenty-four UV light components. In some embodiments, there may be a range of anywhere between ten to thirty UV light components. In some embodiments, there may be an equal amount of UV lighting elements and reflectors such that each UV lighting element has a corresponding reflector. In some embodiments there may be an unequal amount of UV lighting elements and reflectors. Further, in some configurations, the UV light components 120 may be disposed on one or more rows. FIG. 1A illustrates a row of six UV light components 120, there also may be another row of UV light components disposed along side another side of the chamber. In FIG. 1A, the recessed lip of the chamber obscures the six UV light components matching those on the opposite side of the opening of the blowing unit 108. In some configurations, the two rows of UV light components are be disposed along the height of the chamber in a symmetrical arrangement, whereas in other configurations the UV light components in the rows are offset from one another.

In some illustrative configurations, the filter assembly 110 may include one or more components, such as a pre-filter 130, a carbon filter 132, and a high efficiency particulate air filter (HEPA) filter 134 to remove or capture smaller particles. In some configurations, the high efficiency particular air filter removes 99.97% or more of particles 3 microns in diameter. The HEPA filter 134 may include pleating such that the HEPA filter 134 has pleats to increase the surface area of the HEPA filter 134. Increasing the surface area of the HEPA filter 134 may improve the performance and/or the usable life of the HEPA filter 134. The filter assembly 110 may be a flat or substantially flat filter. The filter assembly 110 may have a rectangular shape, although other shapes such as cylinders are considered herein.

In one illustrative embodiment, the HEPA filter 134 may be comprised of a polypropylene (PP) layer melt blown onto a polyethylene terephthalate base layer. In operation, the polypropylene material described herein utilizes, in part, elctromechincal force to entrap, catch, and/or hold contaminants contacting the HEPA filter 134. In yet other embodiments, the HEPA filter 134 also may include, e.g., fiberglass, and/or polytetrafluoroethylene, in addition to or in favor of polypropylene or polyethylene terephthalate (PET).

Though a number of optional configurations may be incorporated, in one illustrative approach, the filter assembly 110 includes a pre-filter 130, carbon filter 132, and a HEPA filter 134. As shown in FIG. 1A, the HEPA filter 134 may be disposed adjacent the UV light components 120. In one illustrative embodiment, the pre-filter 130 may be comprised of mesh such as a nylon mesh material molded or otherwise attached to a plastic frame, such as a screen to entrap contaminants. The pre-filter 130 may be removable from the filter assembly 110 such that a user can replace or clean the pre-filter 130 of any contaminants trapped on or within the pre-filter 130 (e.g., by wiping the pre-filter 130 with a cloth). In some configurations, the carbon filter 132 may be comprised of mesh such as a nylon mesh material molded or otherwise attached to at least two sides of a frame (which may be comprised of a paper material) having extruded activated carbon pellets (EAC) dispersed between the mesh. In some embodiments, the HEPA filter 134 and the carbon filter 132 are combined (such as into one piece or two pieces secured to one another) such that a user can easily replace both the HEPA filter 134 and the carbon filter 132 at the same time.

As illustrated in FIG. 1B, the air purifier 100 includes a second set of the above-described elements on at least a second side of the air purifier 100. FIG. 1B illustrates a backside view of the air purifier 100. Accordingly, the air purifier 100 may include two filter chambers 112a, 112b, 112a is not clearly illustrated in FIG. 1B but corresponds to the structure 112 as illustrated in FIG. 1A, and two filter assembly 110a, 110b. As shown, two opposing sides of the air purifier 100 include a filter cover 124a, 124b, the filter assemblies 110a, 110b, and the filter chamber 112a, and 112b. The filter assembly 110a, 110b, similar to above, includes a pre-filter 130a, 130b, a carbon filter 132a, 132b, and a HEPA filter 134a, 134b. While the filter chambers are illustrated on opposing sides of the air purifier 100, filter chambers may be disposed on non-opposing, i.e., adjacent sides, and/or may be included on more or less than two sides thereof.

In some embodiments, the peripheral reflective surface 114 may be substantially orthogonal to the recessed panel 102 such that the peripheral reflective surface 114 is substantially perpendicular to the recessed panel 102. The peripheral reflective surface 114 may substantially surround the perimeter of the recessed panel 102 or may be disposed primarily along the sides of the filter chamber 112. In some embodiments, the recessed panel 102 may be rectangular in shape. The peripheral reflective surface 114 may be disposed along all four sides of the recessed panel 102 as one homogeneous structure, and in yet other configurations, only two of the sides may have a reflective surface 114 disposed therein or thereon. In some embodiments the peripheral reflective surface 114 may surround the recessed panel 102 on all sides with individual reflective surfaces on one or more sides of the recessed panel 102. The peripheral reflective surface 114, discussed in more detail below, may be comprised of or coated with a reflective material, such as aluminum, aluminum alloys, or other UV/UVC reflective coatings such as polytetrafluoroethylene (PTFE).

In some embodiments, the air purifier 100 may include wheels on a bottom surface of the air purifier 100 to help move the air purifier 100.

FIGS. 2A and 2B, illustrate a bottom cross-sectional perspective view of a UV light component 220 including discrete reflector(s) 221 and LTV lighting element(s) 218. As shown, the UV light component 220 (similar to UV light component 120 described above) is connected to a portion of a recessed panel 202 (similar to recessed panel 102 described above) including a reflector 221 and a UV lighting element 218. The reflector 221 may be disposed adjacent to the recessed panel 202. In some configurations, the reflector(s) 221 are disposed adjacent to an opening 222 in the recessed panel 202. By some approaches, a plurality of reflectors 221 are disposed along a plurality of openings 222 disposed in the recessed panel 202. The UV lighting element 218 may be disposed within and/or adjacent to the opening 222.

In one illustrative embodiment, the reflector 221 and the UV lighting element 218 may comprise one UV light component 220 of an array of UV light components 216. In some embodiments, the reflector 221, one or more posts 226 for mounting and/or supporting the reflector 221, and the UV lighting element 218 may comprise one UV light component 220 of the array of UV light components 216. The array of UV light components 216 corresponds to the underside of the array of UV light components 116, as described above with reference to FIG. 1A.

The reflector 221 may be supported by one or more post 226, or other supporting or mounting structures. The reflector 221 and the one or more post 226 may be one single structure. The reflector 221, the one or most post 226, and/or the array of UV light components 216 may be mounted to the recessed panel 202 using a screw 228 or through the use of other mechanical means such as a nut, a magnet, adhesives, welding, or other mounting structures such as latches.

Similar to the reflective surface 114 above, the discrete reflectors 221 also may be comprised of an aluminum coating or aluminum alloy coating applied to a reflector structure formed of plastic or another material. In one configuration, the base structure is a vacuum metalized part with a thin layer of reflective material. A number of different reflective material application methods are contemplated including for example, a dipped structure application, a sprayed application, and/or a painted application, among others. More specifically, these methods include, e.g., electroplating, electroless plating, physical vapor deposition (PVD), and atomic layer deposition (ALD).

In some configurations a peripheral reflective surface 214 (similar to peripheral reflective surface 114) may be disposed adjacent to the recessed panel 202. A filter assembly 210 (similar to filter assembly 110) may be disposed adjacent to the recessed panel 202. The recessed panel 202 may include a plurality of openings, such as the opening 222, which correspond to the plurality or array of UV light components 216. As shown in FIG. 2B, for example, screws 228a and 228b correspond to a first UV light component in the array of UV light components 216, and similarly, screws 228c and 228d correspond to a second UV light component in the array of UV light components 216.

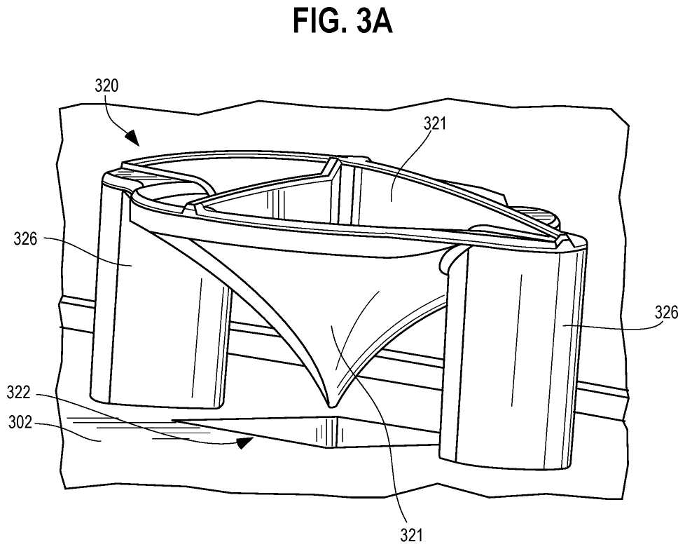

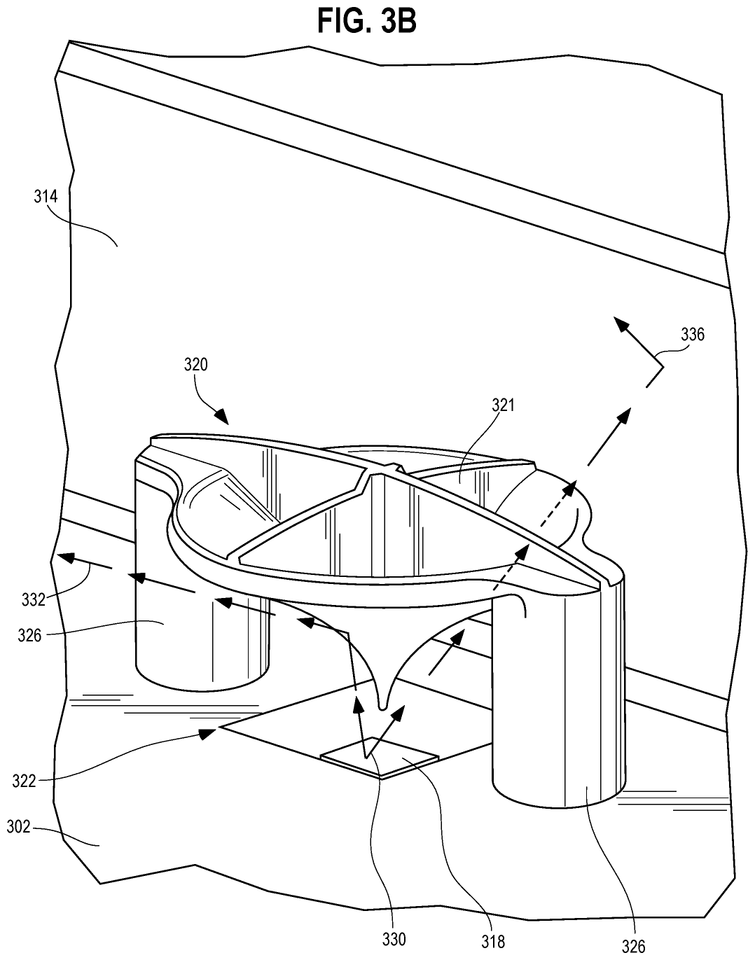

Referring to FIGS. 3A and 3B, a UV light component 320 (similar to those previously described) is illustrated including a reflector 321 having posts 326, and a UV lighting element 318. The UV light component 320 may be disposed on, above, adjacent to, and/or within a recessed panel 302. The recessed panel 302 may include an opening 322. The opening 322 is similar to that described above with reference to FIGS. 1A to 2B, which may permit light emitted or produced from the UV lighting element 318 to pass through the recessed panel 302. The reflector 321 may have a substantially hyperbolic conical shape. In some embodiments, the reflector 321 may have a substantially conical or inverse conical shape. The reflector 321 may be utilized to distribute emitted light across a filter, similar to that described above.

Light emitting from the UV lighting element 318, as illustrated by arrows 330, may pass through the opening 322 of the recessed panel 302. The emitted light 330 may reflect off of, or be diffused by one or more of the reflectors 321, the posts 326, or a peripheral reflective surface 314 prior to irradiating a filter. In addition, at least a portion of the light reflected off the reflector 321, as illustrated by arrow 332 may be reflected and/or diffused towards the filter. A portion of the reflected light 332 may be reflected towards the recessed panel 302. The light emitted from the UV lighting element 318 also may reflect off the peripheral reflective surface 314, as illustrated by arrow 336. In short, some of the light emitted by the UV lighting element 318 is reflected from another surface and onto a surface of the filter. Indeed, in one illustrative approach, a vast majority of the UV light irradiating the filter is reflected from one or more surfaces within the air purifier.

By some approaches, the reflected light 332 and 336 may irradiate the surface of a filter. In some configurations, a portion of the light emitted by the UV lighting element 318, as illustrated by arrows 330, may not be reflected and may irradiate the filter directly. A portion of the light emitted by the UV lighting element 318, as illustrated by arrows 330, may be reflected by the posts 326. A portion of the light emitted by the UV lighting element 318, as illustrated by arrows 330, may be absorbed by one or more of the above-described surfaces.

As described above, the reflectors 321, the posts 326, and the peripheral reflective surface 314, may be utilized to reflect light emitted from the UV lighting element 318 across a surface of a filter assembly, such as the filter assemblies 110 and 210 described above, herein after the filter assembly 110. The following discussion will be focused on a single reflector 321, a single UV lighting element 318, a single pair of posts 326, and a single peripheral reflective surface 314, however an array of UV light components, including an array of corresponding reflectors 321, posts 326, and UV lighting elements 318, as well as a plurality of peripheral reflective surfaces 314 are considered in the present disclosure.

The emitted, diffused, and/or reflected light may irradiate the surface of the filter assembly 110 to inactivate microorganisms. Specifically, in irradiating the surface of the filter assembly 110 microorganisms, captured within or on the surface of the filter, may be inactivated. In utilizing the UV light components 320, the peripheral reflective surface 314, and the reflector 321, light may be evenly and/or sufficiently distributed across the surface of the filter assembly 110. Even and/or sufficient distribution of light may increase the probability of light interacting with the intended surface or target, i.e., the filter assembly 110. Sufficient irradiation may be used to achieve a desired inactivation rate within a desired time period while limiting or preventing the photodegradation of the filter assembly 110.

While the filter assembly 110 may capture microorganisms, contaminants, and/or particles, irradiating the surface of the filter assembly 110 may additionally or alternatively slow, inhibit, and/or prevent the growth of microorganisms on and/or within the filter assembly 110. This may improve the quality of purified and/or cleaned air expelled by the air purifier. This may also slow, inhibit, or prevent the growth of mold on and/or within the filter assembly 110. This may similarly improve the quality of purified and/or cleaned air expelled by the air purifier. Slowing, inhibiting, and/or preventing the growth of microorganisms and/or mold on and/or within the filter may further allow for a more sanitary disposal of a used filter assembly 110, pre-filter 130, carbon filter 132, HEPA filter 134, or a combination thereof that has been removed from the air purifier 100.

Evenly distributing light across the filter assembly 110 may also allow the filter assembly 110 to avoid potential photodegradation from high light intensity from the UV lighting element 318. In some circumstances, this may prevent the reduction in the usable life of the filter assembly 110. The usable life of the filter assembly 110 may be determined or defined by the clean air delivery rate reducing to 50% of the initial value of the filter assembly 110.

UV/UVC wavelength light may be used to irradiate the filter assembly 110. This light may be used to inactivate microorganisms that may be on the surface of the filter assembly 110 or disposed in the pleats of the HEPA filter 134. UVC light includes wavelengths of light ranging from 200 nm to 280 nm. UVC LEDs may be used for the UV lighting element 318. The UVC LEDs may emit or produce light with wavelengths ranging from 200 nm to 280 nm, and in particular at wavelengths around 222 nm, 255 nm, 265 nm, 275 nm, and 285 nm. UVC light emitted with a wavelength about 265 nm and 275 nm is efficient at inactivating microorganisms while not generating ozone. VUV light in the wavelengths 100 nm to 200 nm may be used when the generation of ozone is not a concern. Other UV lighting elements may be used that emit light at a wavelength around 200 nm to 285 nm, for example, UV or UVC LEDs or bulbs, low-pressure mercury vapor or mercury amalgam UVC lamps, or other suitable light sources. Some light sources may be preferred to others depending on the life-expectancy of the UV lighting element to limit replacements and the optical power or strength required.

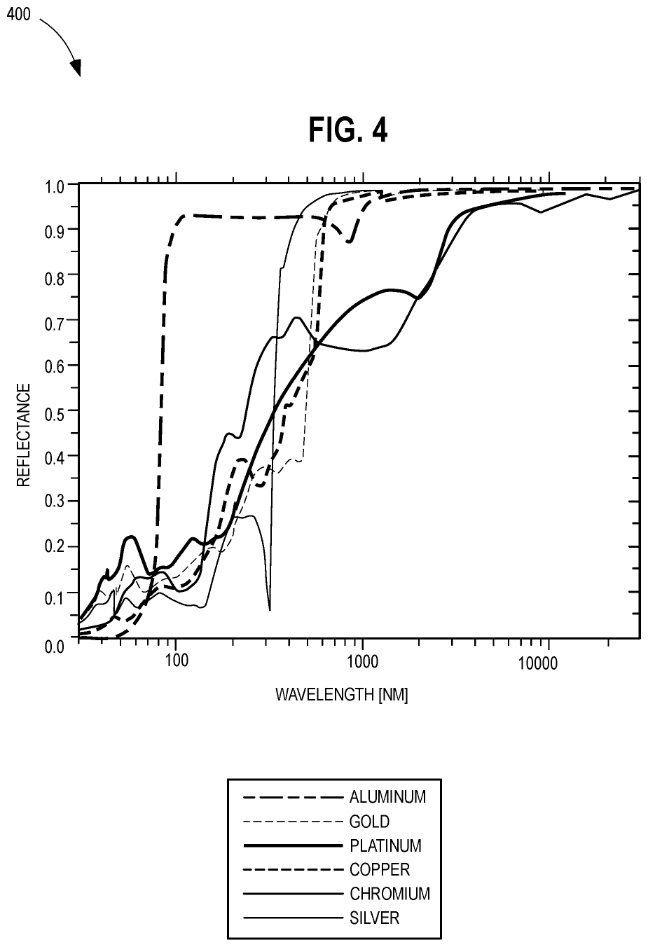

Certain materials may be chosen to reflect light to provide more even distribution of light across the filter assembly 110. Additionally, or alternatively, certain materials may be chosen to provide a high reflectance of UV and/or UVC light having a wavelength around 200 nm to 280 nm. Referring now to FIG. 4, a graph 400 illustrating the reflectivity of different materials on a scale of 0 to 100% reflectivity versus the wavelength of light in nanometers on a logarithmic scale. As shown in the graph 400, around the range of wavelengths of UV and UVC light, around 200 nm to 280 nm, aluminum has a reflectance above 90%. Around the UV and UVC wavelength range, other material such as silver, gold, chromium, copper, and platinum illustrate materials having lower reflectivity as compared to aluminum. Other non-metallic materials with high reflectivity to UVC light, alloys of materials with high reflectivity, and rarer metallic elements with high reflectivity are not illustrated.

The reflector 321, the posts 326, and/or the peripheral reflective surface 314 may be coated or comprised of aluminum or an aluminum alloy to reflect the light emitted by the UV lighting element 318. In some embodiments, the reflector 321, the posts 326, or the peripheral reflective surface 314 may include a coating or be comprised of a reflective material. The reflective material may be aluminum, polished aluminum, an aluminum alloy, or another UV reflective material, such as PTFE, having a reflectivity value of 90% or greater. In some embodiments the reflective material may have a reflectivity value of 95% or greater.

The reflectors 321, the posts 326, or the peripheral reflective surface 314 may be coated with the reflective material, such as aluminum, using a vacuum metallization process. Vacuum metallization is the process of evaporating metal inside a vacuum chamber and bonding the metal to the substrate or base material. In some embodiments electroplating or electrodepositing may be utilized to coat the reflectors 321, the posts 326, or the peripheral reflective surface 314 with the reflective material. Electroplating or electrodepositing is the process of depositing a material, or a reflective material, onto a substrate or base material using electrical current.

In some embodiments physical vapor deposition (PVD) may be utilized to coat the reflectors 321, the posts 326, or the peripheral reflective surface 314 with the reflective material. PVD is the process of producing metal vapor that can be deposited onto a substrate or base material. In some embodiments atomic layer deposition (ALD) may be utilized to coat the reflectors 321, the posts 326, or the peripheral reflective surface 314 with the reflective material. ALD is the process of depositing a coating or film of a material in a vapor phase onto a substrate or base material.

In embodiments utilizing the above processes, techniques, and/or methods to coat the reflectors 321, the posts 326, or the peripheral reflective surface 314, the substrate or base material may be acrylonitrile butadiene styrene (ABS) plastic, or any other suitable material to bond the reflective material coating to the reflectors 321, the posts 326, or the peripheral reflective surface 314.

In some embodiments, the reflectors 321, the posts 326, or the peripheral reflective surface 314 may be a homogeneous structure of the reflective material. A homogeneous structure includes a structure having one similar or uniform characteristic throughout the structure. In some embodiments the homogeneous structure may utilize machining processes, such as the use of a computer numerical controlled (CNC) lathe and/or mill, to shape, form, and/or create the reflectors 321, the posts 326, and/or the peripheral reflective surface 314. In some embodiments the homogeneous structure may utilize stamping from a sheet or piece of reflective material to shape, form, and/or create the reflectors 321, the posts 326, and/or the peripheral reflective surface 314. In some embodiments the homogeneous structure may utilize forming to shape, form, and/or create the reflectors 321, the posts 326, and/or the peripheral reflective surface 314. In some embodiments the homogeneous structure may utilize additive manufacturing, such as 3D printing processes or dual material 3D printing or processes, to shape, form, and/or create the reflectors 321, the posts 326, and/or the peripheral reflective surface 314. In some embodiments the homogeneous structure may utilize casting to shape, form, and/or create the reflectors 321, the posts 326, and/or the peripheral reflective surface 314.

Those skilled in the art will recognize that a wide variety of other modifications, alterations, and combinations can also be made with respect to the above-described embodiments without departing from the scope of the disclosure, and that such modifications, alterations, and combinations are to be viewed as being within the ambit of the inventive concept.

What is claimed is:

1. An air purifier comprising:
a housing having at least one recessed panel disposed inward from an outer surface of the housing and a securement geometry for receiving and securing at least one removable panel to the housing;
an air blowing unit disposed within the housing;
at least one filter disposed between the air blowing unit and the at least one removable panel; and
at least one filter chamber disposed between the at least one removable panel and the recessed panel, the at least one filter chamber including at least one peripheral reflective surface and an array of UV light components, wherein each of the UV light components includes a UV lighting element and one or more discrete reflectors,
wherein light emitted from the UV lighting element is reflected off at least one of the at least one peripheral reflective surface and the one or more discrete reflectors is configured to irradiate the at least one filter.

2. The air purifier of claim 1 wherein the recessed panel includes one or more openings through which light from the UV lighting element is permitted to pass to reflect off at least one of the at least one peripheral reflective surface and the one or more discrete reflectors.

3. The air purifier of claim 2 wherein each of the UV light components include the discrete reflector disposed adjacent the one or more openings.

4. The air purifier of claim 1 wherein each of the UV lighting element emits light at a wavelength around 222 nm to 285 nm.

5. The air purifier of claim 1 wherein each of the UV lighting element emits light at a wavelength around 265 nm to 275 nm.

6. The air purifier of claim 1 wherein each of the UV lighting element comprises a UVC LED, a UV LED, or a low-pressure mercury lamp.

7. The air purifier of claim 1 wherein the one or more discrete reflectors comprise a structure with a hyperbolic conical shape.

8. The air purifier of claim 1 wherein the one or more discrete reflectors comprises a structure with one or more posts.

9. The air purifier of claim 1 wherein the one or more discrete reflectors have an aluminum or aluminum alloy coating.

10. The air purifier of claim 1 wherein the one or more discrete reflectors is a piece of aluminum or aluminum alloy.

11. The air purifier of claim 1 wherein the one or more discrete reflectors comprise a material with a reflectivity value of greater than 90% at UVC wavelengths.

12. The air purifier of claim 1 wherein the one or more discrete reflectors are disposed adjacent the recessed panel.

13. The air purifier of claim 1 wherein the at least one peripheral reflective surface is disposed substantially orthogonal to the recessed panel.

14. The air purifier of claim 1 wherein the at least one peripheral reflective surface has an aluminum or aluminum alloy coating.

15. The air purifier of claim 1 wherein the at least one peripheral reflective surface is a piece of aluminum or aluminum alloy.

16. The air purifier of claim 1 wherein the at least one peripheral reflective surface comprises a material with a reflectivity value of greater than 90% at UVC wavelengths.

17. The air purifier of claim 1 wherein the at least one filter is secured into position between the at least one recessed panel and the removable panel via the securement geometry.

18. The air purifier of claim 1 wherein the UV lighting element comprises of an array of UVC light sources.

19. An air purifying chamber comprising:
a recessed panel disposed adjacent to an air filter;
one or more peripheral reflective surfaces disposed adjacent to a surface of the recessed panel;
an array of UV lighting elements disposed below or partially within the recessed panel; and
a discrete reflector disposed adjacent to each UV lighting element in the array of the UV lighting elements,
wherein the recessed panel includes openings through which light from the UV lighting elements is permitted to pass and the array of the UV lighting elements are disposed adjacent the openings.

20. The air purifying chamber of claim 19 wherein each of the UV lighting elements emits light at a wavelength around 222 nm to 285 nm.

21. The air purifying chamber of claim 19 wherein each of the UV lighting element emits light at a wavelength around 265 nm to 275 nm.

22. The air purifying chamber of claim 19 wherein each of the UV lighting element comprises a UVC LED, a UV LED, or a low-pressure mercury lamp.

23. The air purifying chamber of claim 19 wherein the discrete reflector comprises a structure with a hyperbolic conical shape.

24. The air purifying chamber of claim 19 wherein the discrete reflector comprises one or more posts.

25. The air purifying chamber of claim 19 wherein the discrete reflector has an aluminum or aluminum alloy coating.

26. The air purifying chamber of claim 19 wherein the discrete reflector is a piece of aluminum or aluminum alloy.

27. The air purifying chamber of claim 19 wherein the discrete reflector is disposed adjacent the recessed panel.

28. The air purifying chamber of claim 19 wherein the one or more peripheral reflective surface has an aluminum or aluminum alloy coating.

29. The air purifying chamber of claim 19 wherein the one or more peripheral reflective surface is a piece of aluminum or aluminum alloy.

30. The air purifying chamber of claim 19 wherein the UV lighting elements comprise of an array of UVC light sources.

31. The air purifying chamber of claim 19 wherein the discrete reflector comprises a material with a reflectivity value of greater than 90% at UVC wavelengths.

32. The air purifying chamber of claim 19 wherein the one or more peripheral reflective surface comprises a material with a reflectivity value of greater than 90% at UVC wavelengths.

33. The air purifying chamber of claim 19 wherein the one or more peripheral reflective surface is disposed substantially orthogonal to the surface of the recessed panel.
